(19) United States
(12) Patent Application Publication
(10) Pub. No.: US 2009/0084386 A1
(43) Pub. Date: Apr. 2, 2009

(54) TUBAL LIGATION

(76) Inventors: Annette M.L. McClellan, Heber City, UT (US); Todd S. Parker, Kaysville, UT (US); Karri L. Schlegel, Salt Lake City, UT (US); Steven C. Eror, Salt Lake City, UT (US); Scott B. McClellan, Heber City, UT (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 12/109,638

(22) Filed: Apr. 25, 2008

Related U.S. Application Data
(60) Provisional application No. 60/976,668, filed on Oct. 1, 2007.

(51) Int. Cl. A61F 6/22

(52) U.S. Cl. 128/831; 606/142; 128/843

(57) ABSTRACT

A medical device or system may include an anchor capable of grasping tissue, rotating the tissue, and/or occluding a space in communication with the tissue. A method for occluding a space within a patient may include grasping tissue, rotating the tissue, and/or occluding a space in communication with the tissue.

### CROSS REFERENCE TO RELATED APPLICATIONS

The present application claims the benefit of and priority to U.S. Provisional Patent Application Nos. 60/926,633, filed Apr. 27, 2007, entitled “Tubal Ligator” and 60/976,668, filed Oct. 1, 2007, entitled “Tubal Ligation”, which applications are hereby incorporated by reference in their entirety for all purposes. The present application also hereby incorporates by reference in their entirety for all purposes the following patent application and patents: U.S. patent application Ser. No. 10/846,375, filed May 14, 2004, entitled “Device and Method for Internal Ligation of Tubular Structures”, which application is a continuation of U.S. patent application Ser. No. 10/079,015, filed Feb. 20, 2002, entitled “Device and Method for Internal Ligation of Tubular Structures”, now U.S. Pat. No. 6,737,822, issued May 18, 2004, and U.S. Pat. No. 6,892,682, issued on May 24, 2005, entitled “Method and System for Internal Ligation of Tubular Structures”.

### BACKGROUND OF THE INVENTION

The present disclosure relates to tissue occlusion systems and methods such as tubal ligation systems and methods. Occlusion of tubular anatomical structures is desirable for various medical treatments or intervention. For example, one important application of occlusion techniques is fallopian tube ligation in the female or vas deferens tube ligation in the male to achieve sterilization and prevent undesired pregnancies.

Various methods for producing occlusion or blockage of tubular anatomical structures have been considered for contraceptive and other purposes. A commonly used method for blocking the fallopian tube is to tie off or clamp the fallopian tube using open surgical or laparoscopic surgical approaches. The tube may be tied in two locations and the intermediate portion of the tube removed. A similar result may be obtained by grasping and folding over a portion of the length of the tube and tying off a loop of tube that does not communicate with the remainder of the tube. The folded segment of a tube may be blocked by a loop of suture material, an elastic ligating band, an o-ring, a clip, or a clamp. Access to the fallopian tube is usually gained through endoscopic surgery, either through the abdominal wall or occasionally through the wall of the vagina. Such methods are less invasive than conventional surgical methods but still have an high risk of infection, require anesthesia, cause tissue damage, and are accompanied by an undesirable recovery time and level of discomfort.

To eliminate the need for general surgery or invasive endoscopic or other more invasive surgery, a number of approaches have been devised for blocking the lumen of the fallopian tube after accessing the interior of the fallopian tube by inserting a catheter into the lumen of the tube via the vagina and uterus. One approach is to block the fallopian tube by injecting an adhesive or sealant, typically a polymeric material into the fallopian tube to form a plug. Another approach is to insert a preformed occlusive device or plug into the lumen of the fallopian tube or the uterotubal junction. However, either type of plug may separate or dislodge from the wall of the fallopian tube, resulting in unreliable or impermanent blockage.

Another approach for blocking the fallopian tube or other tubular anatomic structures is to induce the formation of sclerosis or scar tissue to block the tube. Tissue damage may be induced chemically or thermally. However, this method is relatively difficult to accomplish successfully and requires skilled personnel and specialized equipment, making it unsuitable for use in certain settings. Thus, improved systems and methods for occluding and ligating anatomical structures are needed.

### BRIEF SUMMARY OF THE INVENTION

The present invention has been developed in response to problems and needs in the art that have not yet been fully resolved by currently available ligation systems and methods. Thus, among other benefits, these developed systems and methods may provide a method and system for applying a clamping structure to the interior of a tubular anatomical structure, for causing a immediate reliable ligation of a tubular anatomical structure without the need for general surgery or endoscopic surgery, for permanently or reversibly ligating a tubular anatomical structure, for inexpensively ligating a tubular anatomical structure, for employing a partially or completely disposable device for performing ligation of a tubular anatomical structure, for performing tubal ligations through minimally invasive surgery that thereby reduces damage to vascular and reproductive tissues and reduces post-surgical discomfort and recovery time, and/or for performing tubal ligations that further reduce the risk of infection.

A medical device may include an assembly having an axis, an anchor in communication with the assembly, a hinge in communication with the anchor, and/or a surface in opposition to the anchor. The anchor may pivot upon the hinge to move at least a portion of the anchor from the assembly. The anchor may rotate around the axis. The anchor may pivot upon the hinge to move at least a portion of the anchor closer to the surface.

The anchor may include a grasper having a leg, and the leg may include a thigh, a shin, and a knee. The knee may connect the thigh to the shin. The anchor may additionally or alternatively include a spring barb, an elongate structure with a hook, a deployable tent that forms a pointed tip when deployed, metal, alloy, nickel-titanium alloy, stainless steel, and/or a polymer.

The anchor may also include a leading edge. The leading edge is formed to engage with tissue and may include a barb, a tine, a tooth, a hook, a pointed tip, a bristle, a wire, a friction grab, and adhesive, and/or a bump.

The surface in opposition to the anchor may include an additional anchor. The additional anchor may pivot upon an additional hinge to move at least a portion of the additional anchor from the assembly. The additional anchor may also pivot upon the additional hinge to move at least a portion of the additional anchor closer to the anchor.

A medical device may include a distal grasper element, a proximal grasper element, and/or retraction structure. The distal grasper element may be adapted to expand a portion of a cannula in which the distal grasper element is inserted effective to form a fold in the cannula. The proximal grasper element may be adapted to expand a portion of a cannula in which the proximal grasper element is inserted effective to augment the fold. Retraction structure may be adapted to draw the distal grasper element and the proximal grasper element toward each other effective to clamp the fold between the distal grasper element and the proximal grasper element.

The distal grasper element and/or the proximal grasper element may include a leg. The leg may include a thigh, a shin, and/or a knee. The knee may connect the thigh to the shin. One or more thighs may include an axial length greater than the corresponding length of one or more shins.

The distal grasper element and the proximal grasper element may both include multiple legs that are spaced apart evenly and circumferentially around a centerline. The legs of the distal grasper element and the proximal grasper element may be organized into two separate rows of legs including a distal row of legs for the distal grasper element and a proximal row of legs for the proximal grasper element. The distal row of legs may be oriented facing proximally and the proximal row of legs may be oriented facing distally, such that the shins of the distal and proximal grasper elements, when deployed, are in opposition to each other.

A system may include a ligation device and/or an endoscopic tube or cannula for housing a ligation device. The ligation device may include a distal grasper element adapted to expand a portion of a cannula in which the distal grasper element is inserted effective to form a fold in the cannula. The ligation device may also include a proximal grasper element adapted to expand a portion of a cannula in which the proximal grasper element is inserted effective to augment the fold. The ligation device may also include retraction structure adapted to draw the distal grasper element and the proximal grasper element toward each other effective to clamp the fold between the distal grasper element and the proximal grasper element.

The system may also include a trigger adapted to deploy the distal grasper element and the proximal grasper element.

The system may also include a ligation assembly. The ligation assembly may be housed within the endoscopic cannula. The ligation assembly may be separably secured to the ligation device. The trigger may be adapted to disengage the ligation device from the ligation assembly. The trigger may also be adapted to rotate the distal grasper element and the proximal grasper element in relation to each other.

The distal grasper element and/or the proximal grasper element may include a leg. The leg may include a thigh, a shin, and/or a knee. The knee may connect the thigh to the shin. One or more thighs may include an axial length greater than the corresponding length of one or more shins.

The distal grasper element and the proximal grasper element may both include multiple legs spaced apart evenly and circumferentially around a centerline. The legs of the distal grasper element and the proximal grasper element may be organized into two separate rows of legs, including a distal row of legs for the distal grasper element and a proximal row of legs for the proximal grasper element. The distal row of legs may be oriented facing proximally and the proximal row of legs may be oriented facing distally, such that the shins of the distal and proximal grasper elements, when deployed, are in opposition to each other.

A method of manufacturing a medical device may include providing an assembly of a medical device capable of accessing a space within a patient, placing at least one anchor in communication with the assembly, placing at least one surface in communication with the assembly, and/or providing a rotational element between the at least one anchor and the at least one surface. The method of manufacturing may also include laser cutting the at least one anchor. The method of manufacturing may also include biasing the anchor to bend in a direction away from the assembly.

Placing the at least one anchor in communication with the assembly and placing the at least one surface in communication with the assembly may include forming the at least one anchor on a distal end of the assembly and forming the at least one surface proximal to the at least one anchor. Providing a rotational element between the at least one anchor and the at least one surface may include securing the at least one anchor to a first structure and securing the at least one surface to a second structure. The first structure and the second structure may be in rotatable communication with each other. Providing a rotational element between the at least one anchor and the at least one surface may include forming a cannula along the assembly and forming a spiral cut along the cannula of the assembly.

A method of interrupting the continuity of a fluid space may include deploying a first grasper element, deploying a second grasper element, engaging tissue between the first grasper element and the second grasper element, counter-rotating the first grasper element and second grasper element in relation to each other, compressing the tissue either during, after, or independent of counter-rotating, and/or locking the position of the first grasper element and second grasper element in relation to each other. The second grasper element may be a surface, and the second grasper element may be deployed when tissue is placed into contact with the surface as a result of force exerted from the first grasper element. Deploying the first grasper element may include expanding a leg in the direction of tissue, e.g., radially and towards the tissue, including towards the tissue wall. Counter-rotating may include rotating only the first grasper element. The method of interrupting the continuity of a fluid space may also include rotating tissue during, after, or independent of counter-rotating. Locking the position of the first grasper element and second grasper element in relation to each other may include interdigitating the structure of the first grasper element and the second grasper element, engaging a tab with a notch, engaging a pin with hole or depression, engaging a locking mechanism, and/or releasing tension from a loaded locking mechanism by moving the locking mechanism into a locking position.

A method may include accessing the inner portion of a tube having a lumen within a patient, exerting force against the inner surface of the tube, twisting the inner surface of the tube along a longitudinal access, and/or occluding the lumen of the tube while exerting force and twisting. The method may also include locking the lumen of the tube after occluding the lumen of the tube. The method may also include unlocking the lumen of the tube after occluding the lumen of the tube. The tube may include the fallopian tube and/or a structure capable of housing fluid. For example, the tube may include at least one of a uterine or fallopian tube; the vas deferens; any air tube such as the trachea, larynx, pharynx, a bronchus, any bronchial tube or branch, an endobronchial tube, an endotracheal or intratracheal tube, a tracheotomy tube, a nasotracheal tube, an orotracheal tube, Ruysch's tube, Carlen's tube, and Durham's tube; the lungs; any auditory or eustachian tube; a tympanostomy tube; any digestive tube including the esophagus, the large and small intestines, the stomach, a stomach tube, a nasogastric tube, a Cantor tube, a Levin tube, a Miller-Abbott tube, a Moss tube, and a Celestin tube; a nephrostomy tube; a neural or medullary tube; a sphincter; a valve; any blood or other vessel including a lymph vessel, a lymphatic vessel, an afferent vessel, an efferent vessel, a capillary, an anastomosing vessel, and a lacteal vessel; the cardiac tube; any chamber of the heart; a thoracostomy tube; a catheter; a lead having a lumen; a stent; and a drainage tube.

A method may include guiding a medical device to the lumen of the fallopian tube, deploying a tissue anchor of the medical device within the fallopian tube, grasping tissue within the fallopian tube with the tissue anchor, occluding the lumen of the fallopian tube with the tissue anchor, and/or locking the position of the tissue anchor after occluding the lumen of the fallopian tube. The method may also include detaching the medical device from a delivery mechanism. Deploying a tissue anchor may include extending a tissue anchor in the direction of tissue, e.g., extending a tissue anchor radially toward the tissue wall. Grasping tissue with the tissue anchor may include exerting force against the tissue between the tissue anchor and a surface opposing the tissue anchor. The method may also include rotating, clamping, crimping, folding, collapsing, bending, involuting, inverting, and/or plugging tissue within the fallopian tube.

A system may include means for grasping tissue defining a space within a patient, means for rotating the means for grasping, and/or means for occluding the space. The system may also include means for deploying the means for grasping, means for compressing tissue within the space, means for locking the means for grasping, and/or means for accessing the space. Means for grasping may include means for at least one of clamping, crimping, folding, collapsing, bending, involuting, inverting, and/or plugging tissue.

A method may include accessing an implanted medical device for interrupting the continuity of fluid within a space of a patient's body, unlocking a structure of the device, disengaging the device by reversing the action of the initially performed during deployment of the device, and/or removing the device from the patient's body. Unlocking may include breaking the structure. Disengaging may include rotating portions of the device in a direction(s) opposite the direction(s) of rotation initially performed during deployment of the device. Disengaging the device may include disengaging the device from tissue.

These and other features and advantages of the present invention will become more fully apparent from the following description and appended claims or may be learned by the practice of the invention as set forth hereinafter.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is a cross-section view of a device at least partially inserted into a tissue environment.

FIG. 2 is a side view of a medical device.

FIG. 3 is a cross-section view of a grasper assembly within a tissue environment.

FIG. 4 is a cross-section view of a partially deployed grasper assembly within a tissue environment.

FIG. 5 is a cross-section view of a further deployed grasper assembly within a tissue environment.

FIG. 6 is a cross-section view of a still further deployed grasper assembly within a tissue environment.

FIG. 7 is a cross-section view of a rotated grasper assembly within a tissue environment.

FIG. 8 is a cross-section view of a collapsed grasper assembly within a tissue environment.

FIG. 9 is a cross-section view of a deployed grasper assembly within a tissue environment, wherein the grasper assembly is at least partially disengaged from the remaining structure of a medical device.

FIG. 10 is a cross-section view of a deployed grasper assembly within a tissue environment, wherein the grasper assembly is fully disengaged from the remaining structure of a medical device.

FIG. 11 is a partial side view of a grasper element.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the figures herein, could be arranged and designed in a wide variety of different configurations. Thus, the following more detailed description, as represented in the figures, is not intended to limit the scope of the invention as claimed but is merely representative of presently preferred embodiments of the invention.

As used throughout this disclosure, the terms “ligate,” “ligating,” and “ligation” refer to the act of blocking or interrupting the continuity of any tube or other fluid flow space whether by binding, annealing, occluding, or other method of interrupting. Further, as used throughout this disclosure, the terms “tube,” “tubes,” or “tubal” refer to any structure, whether anatomical, manufactured, implanted or implantable, capable of housing fluid. Tubes include without limitation the uterine or fallopian tube; the vas deferens; any air tube such as the trachea, larynx, pharynx, a bronchus, any bronchial tube or branch, an endobronchial tube, an endotracheal or intratracheal tube, a tracheotomy tube, a nasotracheal tube, an orotracheal tube, Ruysch's tube, Carlen's tube, and Durham's tube; the lungs; any auditory or eustachian tube; a tympanostomy tube; any digestive tube including the esophagus, the large and small intestines, the stomach, a stomach tube, a nasogastric tube, a Cantor tube, a Levin tube, a Miller-Abbott tube, a Moss tube, and a Celestin tube; a nephrostomy tube; a neural or medullary tube; a sphincter; a valve; any blood or other vessel including a lymph vessel, a lymphatic vessel, an afferent vessel, an efferent vessel, a capillary, an anastomosing vessel, and a lacteal vessel; the cardiac tube; any chamber of the heart; a thoracostomy tube; a catheter; a lead having a lumen; a stent; and a drainage tube.

Various systems and methods for occluding tubular structures or performing related methods are disclosed in U.S. Pat. No. 5,026,379 to Yoon, issued Jun. 25, 1991; U.S. Pat. No. 5,226,908 to Yoon, issued Jul. 13, 1993; U.S. Pat. No. 5,766,216 to Gangal, et al., issued Jun. 16, 1998; U.S. Pat. No. 5,797,536 to Smith, et al., issued Aug. 25, 1998; U.S. Pat. No. 6,241,740 to Davis, et al., issued Jun. 5, 2001; U.S. Pat. No. 6,527,786 to Davis, et al., issued Mar. 4, 2003; U.S. Pat. No. 6,616,661 to Wellman, et al., issued Sep. 9, 2003; U.S. Pat. No. 6,634,361 to Nikolchev, et al., issued Oct. 21, 2003; U.S. Pat. No. 6,679,892 to Guido, et al., issued Jan. 20, 2004; U.S. Pat. No. 6,736,822 to McClellan, et al., issued May 18, 2004; U.S. Pat. No. 6,758,831 to Ryan, issued Jul. 6, 2004; U.S. Pat. No. 6,896,682 to McClellan, et al., issued May 24, 2005; and U.S. Pat. No. 6,964,274 to Ryan, et al., issued Nov. 15, 2005. These patents are hereby incorporated by reference as a portion of this disclosure as though set forth herein in their entirety. The inventors have identified that the principles and elements of these patents may be applied to the claimed invention.

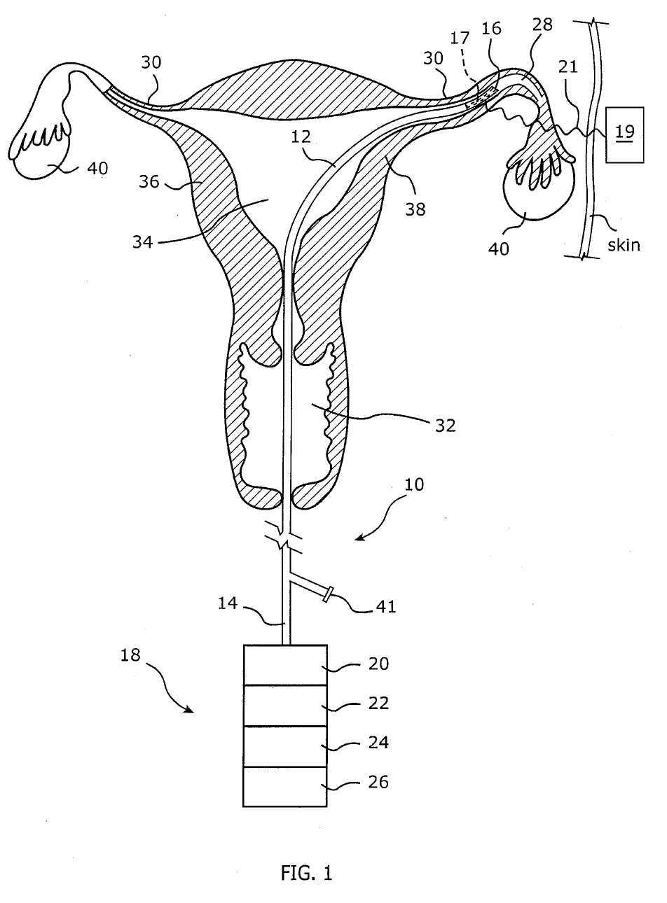

Referring now to FIG. 1, a cross-section view of a medical device 10 is shown at least partially disposed within the anatomy of a patient. The device 10 is used, among other things, to perform internal ligation of tubular structures. Device 10 includes an elongated tubular element 12 having a proximal end 14 and a distal end 16. Proximal end 14 of the tubular element 12 is connected to control segment 18, which includes controls 20, 22, 24, and 28, for controlling the device 10, and which is also used for supporting the device 10 during use. Control segment 18 may be configured as a handle to be held in the hand of a person, such as a medical practitioner, using the device 10, or may be configured for mounting on an examination table or other base or structure. Device 10 is supported and controlled by the control segment 18 while distal end 16 is inserted into the lumen 28 of the fallopian tube 30 of a patient via the vagina 32, lumen 34 of the uterus 36, and uterine horn 38. Ovaries 40 are also shown in FIG. 1. Proximal end 14 may include an access port 41 to permit injection of anesthetics, antibiotics, or other substances into tubular element 12 for infusion into the fallopian tube 30 in the vicinity of the ligation. For example, a radiopaque dye may be infused in the vicinity of the ligation in order to confirm successful blockage of the lumen 28 of the tube 30. The device 10 may also include a gate 17 and an external control device 19 for communicating with and controlling, by means of wired or wireless communications 21, the opening and closing of the gate 17.

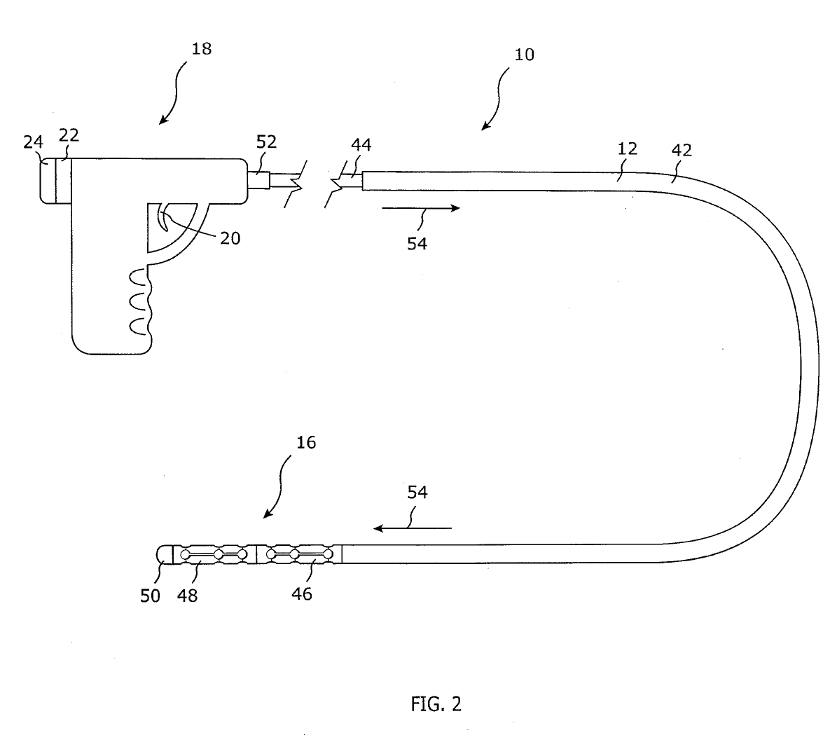

Referring to FIG. 2, a side view of an example of the device 10 is shown. The device 10 has been designed for various procedures including tubal ligation as an office procedure using locally applied topical anesthetics. Device 10 may include an elongated tubular element 12 having an exterior housing, sheath, or structure 42 and an interior housing or structure 44. The interior structure 44 is placed within the exterior structure 42 along the length of the device 10. At the distal end 16 of the device 10, a proximal grasper element 46 and a distal grasper element 48 are formed, for example, in line with the exterior structure 42 such that the proximal grasper element 46 and distal grasper element 48 have substantially the same outer diameter as the exterior structure 42. The proximal grasper element 46 and distal grasper element 48 are held in place by a tip 50 at the distal-most end of the device 10.

The device 10 may also include a control segment 18 having multiple controls 20, 22, 24, and/or other controls. The control 20 may be used as a trigger to deploy a hammer element 52 against the exterior structure 42 such that the exterior structure 42 moves in a distal direction 54 while the interior structure 44 is held in place by the control segment 18. Trigger 20 may also be configured to provide multiple functions such as pushing structures within the device 10, pulling, rotating, crimping, or otherwise manipulating the structure of the device 10 in order to achieve its objectives. For certain medical procedures, the trigger will preferably perform all control functions of the device 10. As the exterior structure 42 moves in a distal direction 54, the proximal grasper element 46 and distal grasper element 48 are deployed. Other controls, such as control 22 and control 24, may be used to rotate either of the elongated structures 42 and/or 44 in relation to each other and/or in relation to the tissue environment. Either of the controls 20, 22, and/or 24 may be used to disengage the proximal and distal grasper elements 46 and/or 48 from the remaining structures of the device 10.

The elongated tubular structure 12 is capable of being placed within the operating channel of a hysteroscope. For example, the structure 12 may be less than 3 mm in diameter. Standard hysteroscope techniques can be used to locate the fallopian tube opening (ostium) and feed the device 10 into the fallopian tube. The device 10 may be placed without hysteroscope equipment effective to provide nonsurgical sterilization options for women in rural or underdeveloped nations. For example, health care providers and/or other professionals with minimal training and/or equipment may provide various helpful procedures to women and other patients using the device 10 with or without a hysteroscope.

When used, for example, to provide sterilization and/or control techniques, a cannula either on the device 10 or used in conjunction with the device 10 may be bent at an angle of approximately 140°. The tip 50 is manually guided through the uterine horn using various tissue channels and/or markers or other imaging equipment and grossly positioned near the uterotubal junction. The device 10 is then advanced from the bent cannula into the ostium and extended about 5 cm. Resistance to insertion during advancement of the device 10 requires the operator to manipulate the tip 50 in search for the tubal opening within the minimal surface area at the tip 50.

Various other guiding structures may be placed at the tip 50 in order to provide further guidance in a particular tissue environment. For example, when searching for the opening of the fallopian tube, a heart-shaped, triangular-shaped, or other relatively bluntly shaped structure may be placed at or near the tip 50 in order to guide the tip 50 toward an end of the uterus likely to include the opening of the fallopian tube. Upon entering the fallopian tube with the tip 50, an operator may verify entry into the fallopian tube by injecting, for example, 20 ml of saline through the length of the device 10 to the tip 50 where saline leakage into the cannula of the device 10, its accompanying structure, or the cervical ostium is indicative of uterine, rather than tubal, tip 50 placement. Various other procedures and/or structures may be employed to ensure appropriate placement of the tip 50 and/or placement of the proximal and/or distal grasper elements 46 and/or 48.

The structures used in connection with device 10 may be used, for example, for sterilization and/or control procedures. Such structures may be formed from extruded nylon, any suitable medical-grade polymer, nitinol, stainless steel, titanium, any metal, any metal alloy, and/or any other biocompatible material. Such structures may be flexible enough to access the desired tissue locations within a patient and rigid enough to provide the necessary structure to provide guidance when accessing certain tissue environments within a patient.

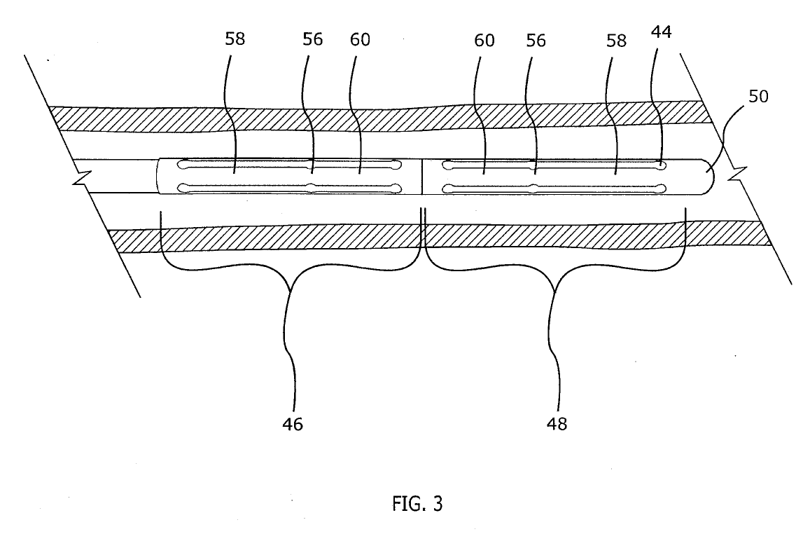

Referring to FIG. 3, a close-up cross-section view of the proximal grasper element 46, distal grasper element 48, and tip 50 is shown within a tissue environment, such as a tube. Each of the proximal grasper element and distal grasper element include a plurality of actuatable structures, such as legs, spaced apart circumferentially around an axial centerline. Each leg includes a knee 56 between a thigh 58 and a shin 60. The thigh 58 and/or shin 60 is a structure capable of actuating upon a hinge, such as knee 56, and may be any structure capable of moving with respect to the interior structure 44 of the device 10. The leg structure of an example of a grasper element may be manufactured by applying micromachining techniques such as laser cutting, stamp cutting, molding, and/or other techniques to a small tubular element. The small tubular element may originally be the exterior structure 42 from which the grasper element may later be separated during manufacturing. The small tubular element may be any cannula of any suitable material, such as a portion of a surgical injection needle.

The legs may be carried by respective proximal and/or distal grasper elements 46 and/or 48 and may be organized into two rows, where the distal row of legs is oriented facing proximally and the proximal row of legs is facing distally such that the shins of deployed grasper elements are in opposition to each other. A leg may be sized such that a thigh 58 has an axial length that is greater than a corresponding axial length of a shin 60. A thigh 58 having a longer length acts as a brace or support to the shin 60 through the hinge or knee 56 to add strength and assist in clamping a fold of tissue in the wall of a tube or other tissue environment. When deployed, the thigh 58 and shin 60 move in a direction toward tissue and away from the axial center of the device 10 such that the knee 56 becomes a point or anchor that is capable of grasping tissue.

In practice, an operator first inserts the device 10 by directing the distal end 16 of the device 10 into a patient and toward the ultimate tissue target environment where the grasper elements 46 and/or 48 are to be deployed. For example, the device 10 may be inserted into the fallopian tube. During insertion, the grasper assembly made of the grasper elements 46 and/or 48 is maintained in an undeployed position. In the undeployed position, the grasper assembly is either continuous, flush, or otherwise oriented to enable the tip 50 and grasper assembly to travel to and safely arrive at the tissue target with minimal trauma to the surrounding tissue along the pathway that the device 10 travels. The grasper assembly may be continuous, flush, or otherwise unobtrusive in relation to the outer diameter of the elongated exterior structure 42 or another catheter placed as a sheath near or over the top of the grasper assembly.

An operator performing a procedure with the device 10 may hold the device by the control segment 18 and insert the distal end 16, for example, into the vagina of the patient and then into the lumen of the uterus. The distal end of the device is then guided via manipulation of the control assembly 18 into a uterine horn and into the lumen of a fallopian tube. Correct placement of the distal end 16 may be determined by monitoring the length of either of the tubular structures 42 and/or 44 inserted after the distal end 16 has passed the uterine horn and entered the fallopian tube, as determined by a change in resistance to insertion. After an operator senses a change in resistance, either of the tubular structures 42 and/or 44 may be further inserted into the fallopian tube at an appropriate depth, for example, of 4 to 5 cm within the fallopian tube. Insertion of the device 10 into the uterus and fallopian tube may also be performed with hysteroscopic guidance. The device 10 may include control wires for steering the distal end 16 or may include other steering mechanisms used with catheters or other associated structure. Such control wires or catheters may include steering control on the control segment 18 used for steering the distal end 16 during insertion.

Once the distal end 16 of the tubular structures 42 and/or 44 has been positioned properly within the fallopian tube, the grasper assembly may be expanded out of continuity with its original position, for example, out of continuous diametric relationship with the exterior structure 42. The grasper assembly may be controlled and deployed by an extension control such as a trigger 20 on the control segment 18. The example of a trigger 20 may cause movement of structure which is mechanically linked to the deployment of the grasper assembly. For example, a tension carrying element may be arranged in harmony with a compression carrying element in communication with the trigger 20. Various other mechanisms, including other triggers, twisting, or other mechanisms or actions, may be devised for causing grasper assembly expansion in a direction toward tissue.

The grasper elements 46 and/or 48 may be deployed by proximal displacement of the interior structure 44 and/or distal displacement of the exterior stricture 42 in relation to each other. Such relative displacement of the exterior structure 42 and interior structure 44 relative to each other will force the knees 56 of the proximal and distal grasper elements 46 and/or 48 to buckle and diffract radially outward toward tissue. The knees 56 radially expand and increase, in this example of a device 10, a diameter of a localized portion of the device 10. Such radial expansion of the knees serves to provide several functions. First, the knees expand in a direction toward tissue which will ultimately be grasped by the device 10. Second, the equal and radial expansion of the knees 56 will serve to center the grasper assembly of the device 10 in a tube of tissue or other similar environment in order to ensure ultimate uniform grasping of the tissue within that environment after the legs are fully deployed. Various other functions are performed as the knees radially expand outward.

A deployed grasper element 46 and/or 48 may form a circumferentially disposed fold of tissue in an environment, such as the fallopian tube. The knee 56 portions of the grasper elements 46 and/or 48 and friction or other force on the tissue walls may permit circumferential grasping of the interior wall of, for example, the fallopian tube at two locations spaced apart along the axis of the distal end 16. As the knees 56 come into contact with tissue, the knees exert force against the tissue and may serve to force the tissue in a direction which will ultimately cause the tissue to be grasped by any structure in connection with a grasper assembly. A folded portion of tissue, such as the fallopian tube, may ultimately be disposed between two grasper elements, or between a grasper element and another structure, to form a tissue bundle. After radially expanding the knees 56, portions of the grasper elements 46 and/or 48 may be drawn together to clamp the tissue bundle between opposing shins 60 or similar structure.

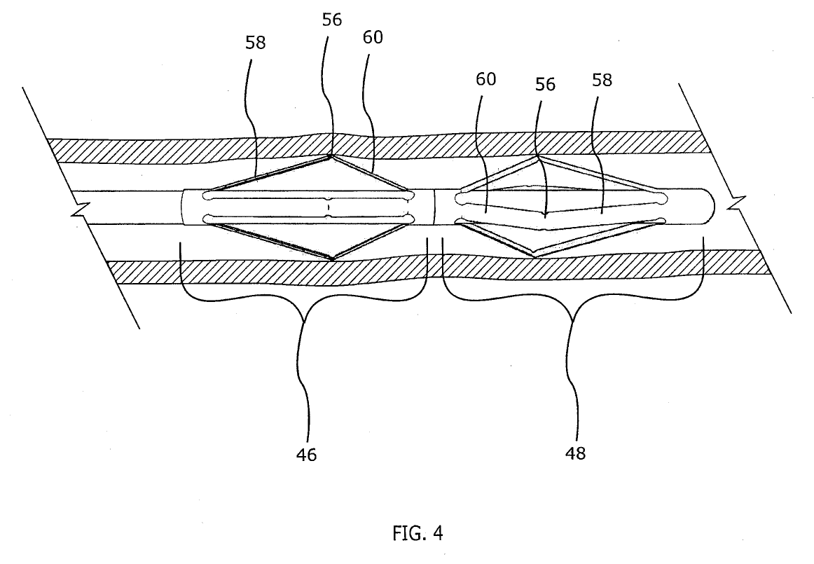

Referring to FIG. 4, a side view of the grasper assembly of the device 10 is shown within a tissue environment. The knees 56 are shown radially extended towards or in the direction of tissue and in contact with the tissue, or tissue wall, as the case may be. The shins 60 are shown having a length that is relatively less than the length of each corresponding thigh 58.

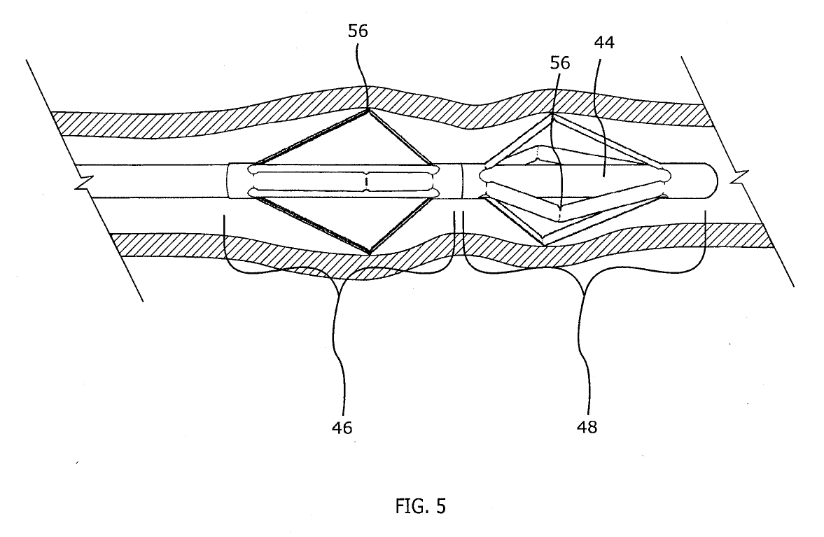

Referring to FIG. 5, the grasper assembly of FIGS. 3 and 4 is shown in side view, with the knees 56 more fully deployed and engaged with surrounding tissue. With the legs more fully deployed on the grasper assembly, the interior structure 44 is more visible.

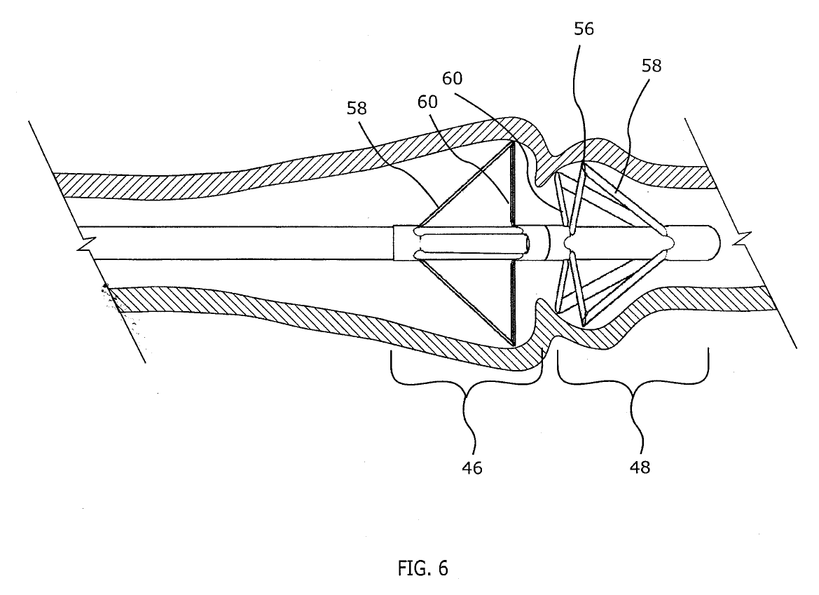

Referring to FIG. 6, a perspective side view of the grasper assembly of FIGS. 3 through 5 is shown with the grasper assembly more fully deployed such that each of the shins 60 is perpendicular to the axis of the device 10. The knees 56 are further extended into surrounding tissue, and the thighs 58 provide support for the knees 56 and shins 60 such that the force exerted by the tissue pinched between the proximal and distal knees 56 does not overwhelm the strength of the supporting thighs 58. With the grasper assembly deployed to a desirable point, for example, such that the shins 60 are perpendicular with the axis of the assembly of the device 10, the proximal and/or distal grasper elements 46 and/or 48 may be rotated, twisted, or otherwise rearranged in relation to each other in order to manipulate the tissue in communication with each grasper element. In one embodiment, the proximal and/or distal grasper elements 46 and/or 48 need not be rotated or twisted in relation to each other, but may merely translate in an axial direction toward or away from each other.

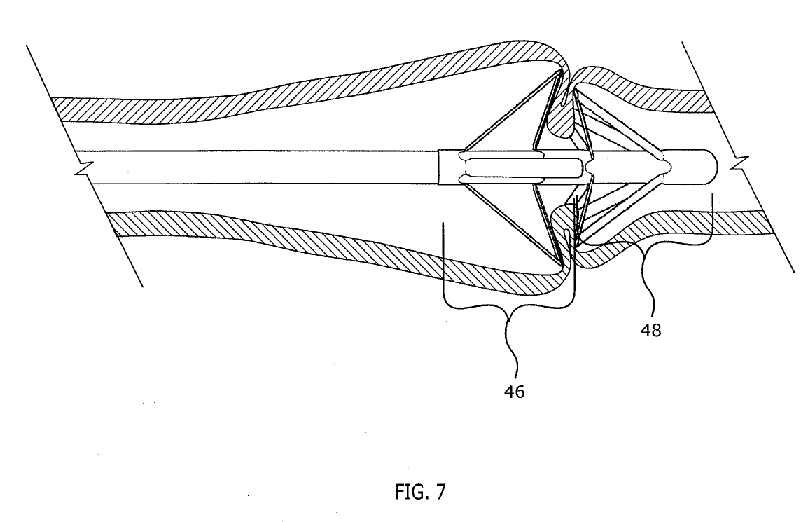

For example, referring to FIG. 7, the proximal grasper element 46 and/or distal grasper element 48 have been rotated in relation to each other in order to pull tissue by means of the anchors or knees 56 in a counter-rotational manner to further constrict the tissue of, for example, the fallopian tube. As tissue is further constricted, the tissue will move closer to the outer diameter of the interior structure 44. When the constricted tissue is in direct contact with the interior structure 44 or the main assembly of the device 10, no gaps within the tissue should exist. Further, by twisting the tissue under a counter-rotational force, the gaps that may exist longitudinally within folds or microfolds of the tissue at any point around the circumference of the device 10 will be compressed and/or folded in a manner which occludes the gaps. Thus a rotational and/or counter-rotational force upon tissue by a grasper assembly or similar structure of a device 10 will serve to occlude and/or interrupt a space within and/or near a tissue environment of a patient.

Any relative rotation and/or other movement may be applied between grasper elements or other structures to cause twisting, compression, rotation, counter-rotation, or other movement of a wall of tissue, such as the fallopian tube, and an overall reduction of the internal circumferential diameter of the tube. For example, such reduction of the internal circumferential diameter of a tube, or other such interruption of a space within a patient, may occur to the point of occlusion of the tube around the outer circumference or outer surface of the elongated tubular structure of the device 10. Any amount of rotation preferred by an operator may be applied. For example, an operator may apply approximately 270° of relative rotation prior to clamping a tissue bundle between grasper elements.

As previously mentioned, as the grasper assembly is expanded from the tubular structure 42, radially expanding the knee 56 portions of the proximal and/or distal grasper elements 46 and/or 48, tissue on the interior of a wall is grasped by the knees 56 which operate as tissue anchors. Such tissue anchors serve to occlude or otherwise interrupt a space by means of rotation, clamping, crimping, folding, collapsing, bending, involuting, inverting, plugging, and/or otherwise anchoring tissue. The control segment 18 (FIGS. 1 and 2) may include a grasp control, such as controls 22 and/or 24 described with reference to FIG. 2 for controlling the grasping of tissue by means of the grasper assembly. Barbs, tines, and/or any other structure known to those of ordinary skill in the art may be incorporated into a grasper element, for example at the knee, to increase the effectiveness of the grasping mechanism or structure in relation to its operation with tissue.

Once tissue has been grasped by the structure of the grasper assembly, a circumferential tissue fold or peduncle will be formed as discussed previously. Such circumferential tissue fold or peduncle may be formed around the entire circumference or surface of the device 10 and/or around only a part of the device 10. After the tissue is adequately grasped by the grasper assembly, the tissue may then be clamped into a permanent and/or semi-permanent clamped tissue bundle.

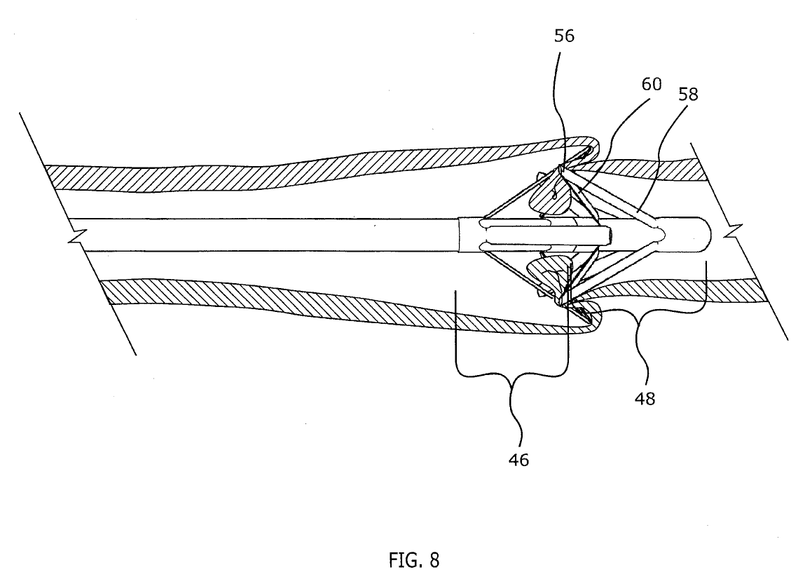

Referring to FIG. 8, a side view of the grasper assembly described with reference to FIGS. 3 through 7 is shown with the grasper assembly clamping a tissue bundle. After the legs of the proximal and distal grasper assemblies 46 and/or 48 have been rotated, at least minimally with respect to each other, the grasper assembly is further deployed such that the shins 60 of both grasper elements 46 and/or 48 have folded beyond a 90° angle with respect to the axial center of the device 10. With the shins extended beyond 90°, the thighs 58 begin to retract or return in a direction toward the axial center of the device 10. As the thighs retract, the knees located at the end of the thighs 58 and shins 60 also retract in a direction toward the axial center of the device 10, pulling compressed and/or bundled tissue in a direction toward the axial center of the device to further occlude any space that may exist between the device 10 and the tissue.

As shown in FIG. 8, the interdigitation and/or interlocking of the knee areas of each respective leg on both the proximal and/or distal grasper elements 46 and/or 48 serve to resist unwinding or unraveling of a rotated grasper assembly. The interdigitation or interlocking of each of the leg assemblies with respect to the other leg assemblies also serves to further occlude the space between the device 10 and the tissue by further wrinkling and contorting the clamped tissue bundle. When the grasper elements are fully engaged and clamped or locked, a structure within the grasper assembly, which may be loaded under spring-loaded tension, may trigger and lock such that the grasper assembly becomes irreversibly and/or reversibly locked. For example, a tab may engage with a notch, a pin may engage with a hole or depression, and/or any other locking mechanism may engage—either automatically or manually under the control of an operator—to lock the position of the leg assemblies in relation to each other. engaging a locking mechanism, and releasing tension from a loaded locking mechanism by moving the locking mechanism into a locking position.

The grasper assembly may be unlocked by further access by the device 10 or any other similar device capable of unlocking or disengaging the locking structure within the grasping assembly. Patients may desire to reverse the lock of the grasper assembly in order to remove the grasper assembly and any other foreign medical devices and/or structure based on future preference and/or medical needs.

In addition to the natural locking mechanism of interdigitation of the legs with respect to each other and the friction and force caused by the compressed tissue between the legs, and in addition to the locking structure previously described, a ratcheting track, e.g., a structure that operates similar to the irreversible operation of a zip-tie, and/or other structure may be employed to resist backward movement of the folded legs of the grasper elements subsequent to rotation that prevents counter-rotation of the grasper assembly. Such backward movement resistant may be reversible. That is, the lock may be opened to enable backward movement and removal of the device 10. Additional or alternative clamping mechanisms that involve independent binding of different aspects of the grasper assembly are also contemplated within the present disclosure. After the clamping mechanism is deployed, for example, the fallopian tube is immediately occluded and capable of providing immediate contraception. Chronic exposure to the deployed grasper assembly may initiate one or more inflammatory responses and result in long-term scar tissue formation, further ensuring tubal occlusion.

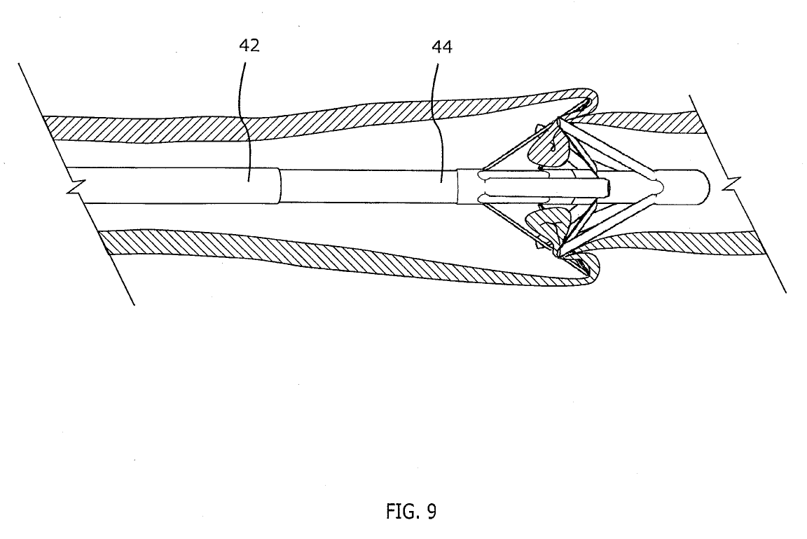

Referring now to FIG. 9, a cross-section view of the grasper assembly described with reference to FIGS. 3 through 8 is shown in side view with a grasped tissue bundle. Following occlusion or ligation of the tissue bundle by deploying a clamping mechanism, the device 10 may be withdrawn, leaving the grasper assembly in place with tissue. After applying a clamp, for example, the lumen of the fallopian tube is divided into two sections separated by the clamp: the distal lumen on the side closer to the ovary and the proximal lumen on the side closer to the uterus. The grasping and/or clamping portions of the device 10 will remain in the fallopian tube as an integral part of any resulting occlusion. The remaining proximal or delivery portion of the device 10 will be detached from the distal or grasping portion of the device 10 prior to removal. Methods for detachment may complement the grasper or clamping device in order to permit optimal efficiency in device delivery and withdrawal. The proximal device is then withdrawn by pulling the external handle or control segment 18 until the non-implantable portion of the device 10 is completely removed from the body of a patient.

For example, as shown in FIG. 9, the external elongated structure 42 is first removed from the grasper assembly by retracting the elongated structure 42 in a proximal direction.

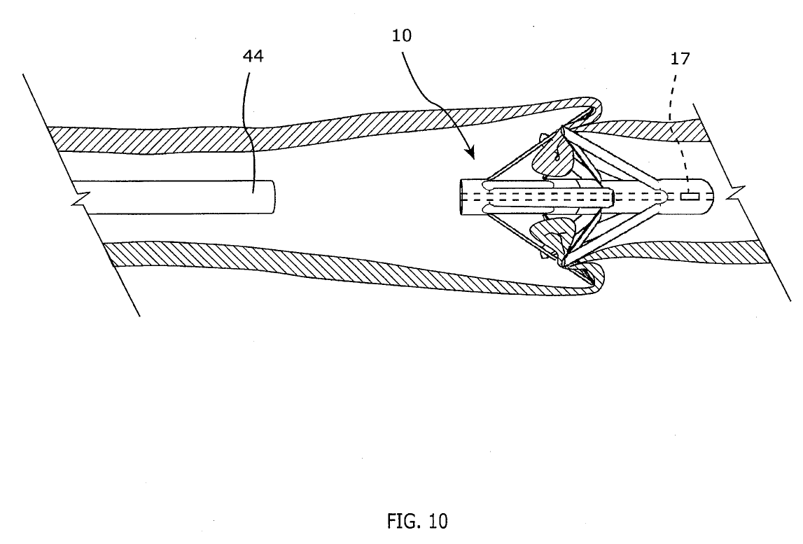

Referring to FIG. 10, a side view of the grasper assembly described with reference to FIGS. 3 through 9 is shown with both the elongated tubular structure 42 and interior structure 44 fully removed and disengaged from the grasper assembly of the device 10. The interior structure 44 and/or other portions of the device 10 may be disengaged from the grasper assembly using zip, flange, button, rotational locks with longitudinal slides, and/or other structures and methods capable of achieving a disengagement function. Further, any of the structures of the device 10 may rotate along a grove in a track advanced by a single trigger, such as trigger 20 described with reference to FIG. 2, in order to form the rotational, engagement, and/or disengagement functions which may be preferred by an operator of a device 10.

If the success of any occlusion and/or interruption of a space within a patient's body is in doubt, it is possible to repeat the previously described procedure within the same or adjacent tissue environment, for example, the fallopian tube at a location that is preferable. For example, within the fallopian tube, the procedure may be repeated at a location that is more proximal from the original deployment location. To accomplish sterilization, it is of course necessary to ligate or clamp both fallopian tubes. Thus, the procedure would be repeated for the second tube in a similar manner. A gate 17 along the axis of the device 10 may provide controlled fluid access and communication between fluid spaces on proximal and distal sides of the device 10.

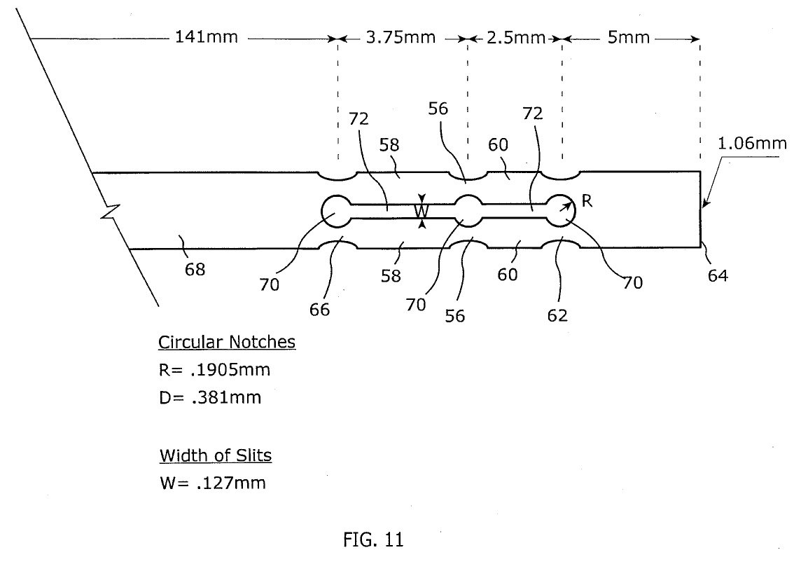

Referring now to FIG. 11, a close-up side view of an example of a grasper element, whether proximal, distal, or otherwise, is shown. In this example, various cuts or spaces have been formed within the material of the grasper element. These spaces form the shape of multiple knees 56, thighs 58, and shins 60. In this example, the length between the end 62 of the shin and the end 64 of the grasper element is approximately 5 mm. The length of the shin is approximately 2.5 mm, the length of the thigh is approximately 3.75 mm, and length from the end 66 of the thigh 58 and remainder of the shaft or tubular element 68 is approximately 141 mm. As previously discussed, the remainder of the tubular shaft 68 may be the exterior structure 42 as previously shown and described. A cut may later be made in the tubular element 68 to form the separate exterior structure 42 (FIG. 2) and grasper element. The tubular element 68 includes a relatively consistent diameter of approximately 1 mm. Any other diameter consistent with the principles of the claimed invention may be employed. Further, the structure of a grasper element could be formed in a cylindrical, square, triangular, regular, irregular, and/or other shape or size as preferred by an operator of the device 10. The spaces defining the leg assemblies may include circular notches 70 that are approximately 0.381 mm in diameter each. The slits 72 between each of the notches 70 may be approximately 0.127 mm wide.

Each of the leg structures may be pre-bent during manufacturing to bias the knees 56 in a radially outward direction. Pre-biasing the leg structures will ensure that the leg structures buckle at the knees 56 when the grasper element needs to be deployed in a tissue environment. The knees 56 and/or any tissue anchor or other structure may be modified to form any other structure capable of engaging and/or communicating with tissue in a manner consistent with the principles of the claimed invention. For example, the side of a knee 56 or other anchor may be pointed, rounded, blunt, angled, cornered, spiked, jagged, or otherwise formed to engage in an effective manner with tissue. The leading edge of an anchor may be formed to engage with tissue in an optimal manner and may include along the leading edge at least one barb, tine, tooth, hook, pointed tip, bristle, wire, friction grab, adhesive, and/or bump. Such structures may be formed during manufacture of the anchor, knee, or other structure on the grasper element using the same or different manufacturing techniques, such as laser cutting, stamp cutting, molding, welding, adhering, or other manufacturing techniques and/or processes.

The grasper elements described above may include additional and/or alternative structures capable of grasping or otherwise communicating with tissue between two structures on the device 10. For example, in one embodiment, the grasper elements may include one or two self-expanding baskets formed of wire similar to the wire structures or formations used to manufacture stents. As another example, grasper elements may include disks capable of expanding, rotating, and/or compressing in a direction toward each other and/or other engaging structures. As another example, grasper elements may include spiral-shaped hooks having leading edges that expand away from the axial tubular structure 42 of the device 10. After grasping tissue, the grasping elements may then be turned in a direction toward the axial center of the device 10, pulling tissue toward the device 10.

Various additional or alternate embodiments to the grasper assembly, grasper elements, and/or anchors may be employed and fall within the scope of the claimed invention. For example, a pre-twisted spiral coil may include multiple barbs, tents, or other anchors on the external circumferential surface of the two ends of the spiral coil. After an external cannula is removed from the external surface of the spiral coil and barbs, the barbs will extend radially toward tissue and the spiral coil will unwind causing the two ends of the barbs to move closer together grasping and rotating tissue as they move toward each other. Ultimately, the barbs will pull a tissue bundle or peduncle toward the surface of the spiral coil as it expands, causing the space between the grasper assembly and the tissue to become at least partially occluded.

As another example, a grasper assembly may include two structures spaced within a tube or other area of tissue within a patient's body. Each of the two structures will be either inflated or placed into contact with all areas of tissue surrounding the structures, causing the space between the structures and the tissue to be totally sealed or occluded. A structure may then be used to remove air or fluid from between the two structures, causing a negative pressure in the chamber formed between the two structures and the surrounding tissue. As the pressure within the chamber decreases, the tissue between the two sealing structures will collapse toward the structure removing fluid from the space. As the tissue collapses, the space between the two sealing structures will become further occluded, decreased, and/or interrupted.

As yet another example, a grasper assembly may include a spring and/or a spiral coil capable of rotating along the axial length of a tissue structure forming a tube. The coil will include two ends having larger loops and a center having smaller loops of the coil. As the coil is spun along the axial length of the tube in a corkscrew motion, a sharp tip or end of the large loops of the coil will pierce through the wall of the tube of tissue. The remainder of the coil will then follow the path of the tip which has pierced through the wall of the tissue, causing the larger loops to sequentially decrease in diameter toward the smaller loops until the smaller loops have penetrated or stitched through the wall of the tube of tissue. As the smaller loops penetrate or stitch into the wall of tissue, the smaller loops will pull the wall of tissue toward the axial center of the coiled loops, causing the walls of the tube to collapse, occlude, and/or otherwise interrupt the fluid space within the tube.

As another example, a grasper assembly may include the manual twisting of two barbs or other anchors in opposite directions by means of a twist handle. As another example, a grasper assembly may include the manual twisting of one barb or anchor while another structure is held in place, causing tissue to be trapped between the one barb or anchor and the additional structure.

As another example, a grasper assembly may include a tent-type structure that deploys similar to the legs previously shown and described. The tent-like structure may operate similar to a toggle bolt which deploys causing the knees to buckle and extend radially outward as the toggle bolt within the tent is rotated. Additionally or alternatively, the tent may simply be compressed causing the knees of the tent to buckle and extend in a radially outward direction. The deployed tent will then grasp tissue and force the tissue in a direction toward another structure such that the tissue is grasped between the additional structure and the expanding tent structure. The tent structure and/or additional structure may include any type or number of anchors capable of providing further communication between the structure and the tissue. The two or more legs of a tent structure may be the same length and/or different lengths. For any embodiment described herein, the angles and surfaces of any anchor described herein may be employed and adjusted with respect to a particular embodiment to either increase the strength of contact between the anchors and engaging tissue or to decrease the amount of contact and limit trauma caused to the tissue as a result of a more gentle contact between the structures and the tissue.

As another example, a grasper assembly may include at least one inflating balloon which inflates and twists the structure of the balloon simultaneously. The inflating and twisting motion causes the walls of tissue within a space to come into contact with the structure of the balloon and twist as a result of the twisting or rotating motion of the structure of the balloon. As the walls of the tissue twist, the space between the balloon structure and the tissue is further occluded and the pressure between the structure and the tissue prevents any fluid access through space between the structure and the tissue. Inflating balloons may include one or more balloons, either of which may rotate during inflation. As another example, one or more silicone, adhesive, or other plugs may be employed to fill a space in conjunction with any of the embodiments described herein. As another example, expanding baskets of wire may operate similar to an expanding balloon to come into contact with and/or rotate the tissue forming the walls of a fluid space.

For use within the fallopian tube, the diameter of the device 10 may be 1 mm (3 French) or less. For use within other tubal structures, such as larger blood vessels, the diameter of the device 10 may be greater than 1 mm. Any structure described herein may include visualization elements such as radiopaque dots or markers on catheters, cannula, anchors, grasper assembly and/or elements, or any other structure described herein. Such radiopaque or other visualization characteristics will assist operators in implantation, deployment, explanation, and other steps involved in the operation of the device 10. Visualization or confirmation of any structure of the device 10 may include imaging techniques, ultrasound, tactile techniques, x-ray, Doppler, or other techniques. Fluid infusion, such as saline, radiopaque dye, and/or air pressure infusion may be used and provided through any structure of the device 10 in order to confirm proper placement and/or provide other information helpful to the particular procedure employing device 10.

The method of deploying the device 10 will, as previously discussed, preferably be placed within 4 to 5 cm past the cervical ostium. In deployment, the grasper assembly will be both transcervical and extramural, enabling the implantation of the grasper assembly of the device 10 to be reversible. A patient desiring to remove the device from the patient's body may request an operation either transecting the tube and tissue in communication with the grasper assembly or the patient may request an operator to unlock the grasper assembly and attempt to remove via endoscopic or other similar techniques. Transection procedures may require that the tube or other tissue remaining, once the grasper assembly has been removed, be sown or otherwise reconnected in order to preserve the continuity of the fluid space in the tissue environment. For example, within the fallopian tubes, a transection procedure will permit the extramurally implanted grasper assembly to be removed, the fallopian tube to be rejoined, if desired, and a new grasper assembly may be implanted elsewhere within the fallopian tube including at or near the muscle wall of the tube.

Every embodiment discussed herein may be configured to be reversible or controllable such that a patient may resume at least minimal function of tissue areas after at least a portion of device 10 has been implanted within the patient. Such reversibility or control may be a desired feature in order to provide temporary contraception, control, and/or occlusion of fluid spaces within the patient.

Control will enable a patient, in certain embodiments, to open and close a fluid space at least to a minimal degree at will. The opening and closing of a fluid space will provide a therapeutic benefit to the patient enabling the patient to change his or her lifestyle as a result of the control. Control will be effected by electromagnetic gates, mechanical gates, chemical gates, and/or other mechanisms of action capable of communicating from the exterior of a patient's body to the implanted device within the patient's body.

For example, a reversible gate may be controlled by means of an external control system capable of opening, closing, and otherwise controlling the reversible gate within the device 10. The gate may be located within the device, such that fluid may flow through the gate when it is opened, passing from one side of the device 10 through the gate to the other side of the device 10. The control system may communicate with the gate, or other control circuitry within the device 10 that controls the gate, by means of radio frequency (RF) telemetry, magnetic communication, a wired connection, an optical connection, or another transcutaneous communication link. Various means of communication between an external control device and implanted medical devices are described with reference to pacing, drug delivery, defibrillator, electrical stimulation, and other implantable systems. For example, U.S. Pat. No. 6,243,608 to Pauly, et al., issued Jun. 5, 2001, describes an implantable device that communicates with an external controller by means of optical telemetry, which patent is incorporated herein by reference in its entirety.

Various conventional structures and/or techniques involved in occlusion and/or ligation of tubal or other tissue structures in a patient's body may be performed. For example, a FILSHIE® clip and/or a Hulke clip may be applied to such tissue structures involved and discussed herein. Various tissue clamping techniques may be combined with the principles discussed herein. As previously mentioned, plugs may be employed with the principles discussed herein.

Further, the metallic and/or other substances may be applied to any structure discussed herein or infused through any structure discussed herein in order to provide irritants or other chemical catalysts which cause certain effects upon tissue. For example, an irritant may be applied to the external surface of a grasper assembly in order to promote cell death, inflammation, scarring, chemical rejection, thermal rejection, oblation, irritation, and/or any other desired effect to promote ligation, occlusion, and/or other interruption of fluid spaces within a patient's body. Further, various chemicals and/or other coatings may be applied to any surface of the structures described herein and/or may be infused through any structure described herein, including coatings that promote tissue growth, are caustic, promote healing, promote treatment of tissue, increase the ease of insertion of certain structures, provide pain relief, and/or provide other helpful and/or essential functions as provided herein.

The present invention may be embodied in other specific forms without departing from its structures, methods, or other essential characteristics as broadly described herein and claimed hereinafter. For example, the elements discussed above may be combined in any number and orientation in an enabling manner with any number and orientation of any of the other elements discussed above to produce various ligation systems and methods. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

1. A medical device, comprising:
an assembly having an axis;
an anchor in communication with the assembly;
a hinge in communication with the anchor; and
a surface in opposition to the anchor;
wherein the anchor pivots upon the hinge to move at least a portion of the anchor from the assembly;
wherein the anchor rotates around the axis; and
wherein the anchor pivots upon the hinge to move at least a portion of the anchor closer to the surface.

2. The medical device of claim 1, wherein the anchor includes a grasper having a leg; wherein the leg includes a thigh, a shin, and a knee; and wherein the knee connects the thigh to the shin.

3. The medical device of claim 1, wherein the anchor includes a spring barb.

4. The medical device of claim 1, wherein the anchor includes an elongate structure with a hook.

5. The medical device of claim 1, wherein the anchor includes a deployable tent that forms a pointed tip when deployed.

6. The medical device of claim 1, wherein the anchor includes a nickel-titanium alloy.

7. The medical device of claim 1, wherein the anchor includes a polymer.

8. The medical device of claim 1, wherein the anchor includes a leading edge; wherein the leading edge is formed to engage with tissue; and wherein the leading edge includes at least one of a barb, a tine, a tooth, a hook, a pointed tip, a bristle, a wire, a friction grab, an adhesive, and a bump.

9. The medical device of claim 1, wherein the surface in opposition to the anchor includes an additional anchor, wherein the additional anchor pivots upon an additional hinge to move at least a portion of the additional anchor from the assembly, and wherein the additional anchor pivots upon the additional hinge to move at least a portion of the additional anchor closer to the anchor.

10. A medical device, comprising:
a distal grasper element adapted to expand a portion of a tube in which said distal grasper element is inserted effective to form a fold in said tube;
a proximal grasper element adapted to expand a portion of a tube in which said proximal grasper element is inserted effective to augment said fold; and
retraction structure adapted to draw said distal grasper element and said proximal grasper element toward each other effective to clamp said fold there-between.

11. The medical device of claim 10, wherein the distal grasper element includes a leg, wherein the leg includes a thigh and a shin with a knee, and wherein the knee connects the thigh to the shin.

12. The medical device of claim 11, wherein the proximal grasper element includes a leg, wherein the leg includes a thigh and a shin with a knee, and wherein the knee connects the thigh to the shin.

13. The medical device of claim 12, wherein the thighs include an axial length greater than the corresponding length of the shins.

14. The medical device of claim 13, wherein the distal grasper element and the proximal grasper element both include multiple legs spaced apart evenly and circumferentially around a centerline, wherein the legs of the distal grasper element and the proximal grasper element are organized into two separate rows of legs including a distal row of legs for the distal grasper element and a proximal row of legs for the proximal grasper element.

15. The medical device of claim 14, wherein the distal row of legs is oriented facing proximally and the proximal row of legs is oriented facing distally such that the shins of the distal and proximal grasper elements, when deployed, are in opposition to each other.

16. A system, comprising:
an endoscopic cannula for housing a ligation device; and
a ligation device, including:
a distal grasper element adapted to expand a portion of a tube in which said distal grasper element is inserted effective to form a fold in said tube;
a proximal grasper element adapted to expand a portion of a tube in which said proximal grasper element is inserted effective to augment said fold; and
retraction structure adapted to draw said distal grasper element and said proximal grasper element toward each other effective to clamp said fold there-between.

17. The system of claim 16, further comprising a trigger adapted to deploy the distal grasper element and the proximal grasper element.

18. The system of claim 17, further comprising a ligation assembly, wherein the ligation assembly is housed within the endoscopic cannula, wherein the ligation assembly is separably secured to the ligation device, and wherein the trigger is adapted to disengage the ligation device from the ligation assembly.

19. The system of claim 18, wherein the trigger is adapted to rotate the distal grasper element and the proximal grasper element in relation to each other.

20. The system of claim 19, wherein the distal grasper element includes a leg, wherein the leg includes a thigh and a shin with a knee, and wherein the knee connects the thigh to the shin.

21. The system of claim 20, wherein the proximal grasper element includes a leg, wherein the leg includes a thigh and a shin with a knee, and wherein the knee connects the thigh to the shin.

22. The system of claim 21, wherein the thighs include an axial length greater than the corresponding length of the shins.

23. The system of claim 22, wherein the distal grasper element and the proximal grasper element both include multiple legs spaced apart evenly and circumferentially around a centerline, wherein the legs of the distal grasper element and the proximal grasper element are organized into two separate rows of legs including a distal row of legs for the distal grasper element and a proximal row of legs for the proximal element.

24. The system of claim 23, wherein the distal row of legs is oriented facing proximally and the proximal row of legs is oriented facing distally such that the shins of the distal and proximal grasper elements, when deployed, are in opposition to each other.

25. A method of manufacturing a medical device, comprising:
providing an assembly of a medical device capable of accessing a space within a patient;
placing at least one anchor in communication with the assembly;
placing at least one surface in communication with the assembly; and
providing a rotational element between the at least one anchor and the at least one surface.

26. The method of claim 25, further comprising laser cutting the at least one anchor.

27. The method of claim 25, wherein placing the at least one anchor in communication with the assembly and placing the at least one surface in communication with the assembly include forming the at least one anchor on a distal end of the assembly and forming the at least one surface proximal to the at least one anchor.

28. The method of claim 25, wherein providing a rotational element between the at least one anchor and the at least one surface includes securing the at least one anchor to a first structure and securing the at least one surface to a second structure, wherein the first structure and the second structure are in rotatable communication with each other.

29. The method of claim 25, wherein providing a rotational element between the at least one anchor and the at least one surface includes forming a cannula along the assembly and forming a spiral cut along the cannula of the assembly.

30. The method of claim 25, further comprising biasing the anchor to bend in a direction away from the assembly.

31. A method of interrupting the continuity of a fluid space, comprising:
deploying a first grasper element;
deploying a second grasper element;
engaging tissue between the first grasper element and second grasper element;
counter-rotating the first grasper element and second grasper element in relation to each other;
compressing the tissue; and
locking the position of the first grasper element and second grasper element in relation to each other.

32. The method of claim 31, wherein the second grasper element is a surface, and wherein the second grasper element is deployed when tissue is placed into contact with the surface as a result of force exerted from the first grasper element.

33. The method of claim 31, wherein deploying the first grasper element includes expanding a leg towards the tissue.

34. The method of claim 31, wherein counter-rotating includes rotating only the first grasper element.

35. The method of claim 34, further comprising rotating tissue during counter-rotating.

36. The method of claim 35, wherein locking the position of the first grasper element and second grasper element in relation to each other includes at least one of interdigitating the structure of the first grasper element and the second grasper element, engaging a tab with a notch, engaging a pin with hole or depression, engaging a locking mechanism, and releasing tension from a loaded locking mechanism by moving the locking mechanism into a locking position.

37. A method, comprising:
accessing the inner portion of a tube having a lumen within a patient;
exerting force against the inner surface of the tube;
twisting the inner surface of the tube along a longitudinal axis; and
occluding the lumen of the tube while exerting force and twisting.

38. The method of claim 37, further comprising locking the lumen of the tube after occluding the lumen of the tube.

39. The method of claim 38, further comprising unlocking the lumen of the tube after occluding the lumen of the tube.

40. The method of claim 37, wherein the tube includes the fallopian tube.

41. The method of claim 37, wherein the tube includes a structure capable of housing fluid.

42. The method of claim 41, wherein the tube includes at least one of a uterine or fallopian tube; the vas deferens; any air tube such as the trachea, larynx, pharynx, a bronchus, any bronchial tube or branch, an endobronchial tube, an endotracheal or intratracheal tube, a tracheotomy tube, a nasotracheal tube, an orotracheal tube, Ruysch's tube, Carlen's tube, and Durham's tube; the lungs; any auditory or eustachian tube; a tympanostomy tube; any digestive tube including the esophagus, the large and small intestines, the stomach, a stomach tube, a nasogastric tube, a Cantor tube, a Levin tube, a Miller-Abbott tube, a Moss tube, and a Celestin tube; a nephrostomy tube; a neural or medullary tube; a sphincter; a valve; any blood or other vessel including a lymph vessel, a lymphatic vessel, an afferent vessel, an efferent vessel, a capillary, an anastomosing vessel, and a lacteal vessel; the cardiac tube; any chamber of the heart; a thoracostomy tube; a catheter; a lead having a lumen; a stent; and a drainage tube.

43. A method, comprising:
guiding a medical device to the lumen of the fallopian tube;
deploying a tissue anchor of the medical device within the fallopian tube;
grasping tissue within the fallopian tube with the tissue anchor;
occluding the lumen of the fallopian tube with the tissue anchor; and
locking the position of the tissue anchor after occluding the lumen of the fallopian tube.

44. The method of claim 43, further comprising detaching the medical device from a delivery mechanism.

45. The method of claim 44, wherein deploying a tissue anchor includes extending a tissue anchor radially toward the tissue wall.

46. The method of claim 45, wherein grasping tissue with the tissue anchor includes exerting force against the tissue between the tissue anchor and a surface opposing the tissue anchor.

47. The method of claim 43, further comprising at least one of the following steps:
rotating tissue within the fallopian tube;
clamping tissue within the fallopian tube;
crimping tissue within the fallopian tube;
folding tissue within the fallopian tube;
collapsing tissue within the fallopian tube;
bending tissue within the fallopian tube;
involuting tissue within the fallopian tube;
inverting tissue within the fallopian tube;
removing at least a portion of the medical device from a patient;
removing a non-implantable portion of the medical device from a patient;
removing an insertion catheter portion of the medical device from a patient; and
plugging tissue within the fallopian tube.

48. A system, comprising:
means for grasping tissue defining a space within a patient;
means for rotating the means for grasping; and
means for occluding the space.

49. The system of claim 48, further comprising means for deploying the means for grasping.

50. The system of claim 48, further comprising means for compressing tissue within the space.

51. The system of claim 48, further comprising means for locking the means for grasping.

52. The system of claim 48, further comprising means for accessing the space.

53. The system of claim 48, wherein means for grasping includes means for at least one of clamping, crimping, folding, collapsing, bending, involuting, inverting, and plugging tissue.

54. A method, comprising:
accessing an implanted medical device for interrupting the continuity of fluid within a space of a patient's body;
unlocking a structure of the device;
disengaging the device by reversing the action of the initially performed during deployment of the device; and
removing at least a portion of the device from the patient's body.

55. The method of claim 54, wherein unlocking includes breaking the structure.

56. The method of claim 54, wherein disengaging includes rotating portions of the device in a direction opposite the direction of rotation initially performed during deployment of the device.

57. The method of claim 54, wherein disengaging the device includes disengaging the device from tissue.
